(19) United States
(12) Patent Application Publication
(10) Pub. No.: US 2008/0086076 A1
(43) Pub. Date: Apr. 10, 2008

(54) Anti-aspiration device with content monitoring functionality

(76) Inventor: Allen Gerber, High Falls, NY (US)

Correspondence Address: Lawrence D. Cutter, 11 Deer Path Drive, New Paltz, NY, 12561-2813, US

(21) Appl. No.: 11/804,109

(22) Filed: May 17, 2007

Related U.S. Application Data
(63) Continuation-in-part of application No. 11/545,382, filed on Oct. 10, 2006.

(51) Int. Cl. A61M 39/00

(52) U.S. Cl. 604/43; 604/19

(57) ABSTRACT

A patient stomach fullness sensor is employed in conjunction with an optional patient angle sensor to shut off or to reverse the flow of fluid in a gastric tube when the combination of stomach fullness and patient angle relative to the horizontal becomes sufficient to indicate that gastric juices may enter the esophagus or go even higher. In this way incidents of aspirational pneumonia in hospitalized patients is significantly reduced or eliminated.

### CROSS-REFERENCE TO RELATED APPLICATIONS

The Present application is a continuation-in-part application of patent application Ser. No. 11/545,382 filed on Oct. 10, 2006. This application contains subject matter which is related to the subject matter of the above-mentioned application, which is owned by the same entity as this application.

### TECHNICAL FIELD

The present invention is generally directed to the medical field as it relates to patient care, particularly in a hospital, nursing home or other institutional setting. More particularly, the present invention relates in general to systems and methods for preventing aspiration of stomach contents by bedridden patients connected to feeding tubes. Even more particularly, the present invention is directed to systems and methods for determining the level and/or relative quantity of stomach contents in situations in which a patient is receiving nutrition through a feeding tube.

### BACKGROUND OF THE INVENTION

It is well known that millions of people around the world are fed through gastric feeding tubes once they can no longer feed themselves. The most common version of this practice occurs in the use of nasogastric feeding tubes. Other gastric feeding practices include the surgical insertion of a feeding tube directly into the stomach through the abdominal wall (PEG tube). The present invention is employable in all of these situations in which gastric feeding is provided, though some embodiments are more appropriate for the PEG tube situation.

While the use of gastric feeding mechanisms is not only a common but a life preserving procedure, complications can arise. In particular, one of these complications is aspiration pneumonia. This condition, which can be life threatening, particularly in older patients or in patients with weakened immune systems, can occur via several mechanisms. A common one of these mechanisms is one in which the patient slides down in bed to a low angle sufficient to allow gastric fluids to ascend the esophagus and be inhaled into the lungs. Typically, this angle is about 30°. When the patient angle in the bed reaches this point, the stomach contents are able to percolate up through the esophagus and down into the lungs. The fact that this is a significant problem in patient care is reflected in the fact that in many states the occurrences of aspirational pneumonia are reportable incidents to state oversight authorities, particularly in the case of patient death.

The previously filed application for which this is a continuation in part addressed the problem of aspirational pneumonia with a view toward the triggering mechanism being the patient angle relative to the horizontal. In particular, it was seen that patient angle was a significantly more critical parameter than bed angle. It is noted that the present invention is directed to a related problem, namely the degree to which the stomach contents fill the available stomach volume. With this added piece of information, the previously described, angle-related invention is improved. Thus, in addition to patient angle relative to the horizontal, it is seen that it is also desirable to be able to determine the current degree of stomach fullness.

Whether it is patient angle or the quantity of material present in the stomach, it is the goal of the present invention to prevent stomach contents from rising into the esophageal region. The aforementioned parent application provided two response modalities for addressing the problem of low patient angle. In one modality, flow of feeding material is stopped. In another modality, stomach contents are actually withdrawn through the feeding tube. The present invention adds the dimension of knowing the quantity and/or level of the stomach contents, either in relative or absolute terms. It is noted that reference herein to relative stomach content is a measure of fullness percentage. The present invention works in conjunction with the mechanisms employed in the earlier application, which considered only patient angle as the determining factor in the initiation of a stop or withdraw action. That is to say, the present invention is employable in an embodiment in which the flow control mechanism of the prior invention is employed without regard to patient angle. However, it is noted that the present invention is also employable in various preferred embodiments in which the stomach fullness indicator is employed along with the angle indicator to better control the functioning of the flow control mechanism.

It is noted that, while the present invention is principally directed to the problems associated with gastric feeding tubes, nonetheless, it is equally applicable to those situations in which substances other than nourishment are being provided through such a tube. It is also noted that the inventions described herein have at least two desirable effects. Not only does the present invention and its predecessor, work to prevent aspirational pneumonia, they also work to eliminate or reduce the presence of gastric fluid in the esophagus.

### SUMMARY OF THE INVENTION

The shortcomings of the prior art are overcome and additional advantages are provided through the inclusion of all of one or more other devices or methods used to determine stomach fullness. For example, an ultrasound measurement is employable as providing an indication that sufficient quantities of nutrient or medication have been delivered to the stomach. Likewise, an in situ indicator of stomach gas pressure is also employable as providing an indication that fluid flow should either be stopped or reversed. It is also noted that since girth increases with the degree to which the stomach is filled, it is seen that the simple expedient of providing a girth sensor is also employable as providing a mechanism for determining the flow control.

The invention described in the related prior application cited above solves these problems by providing a mechanical or electronic device that senses when a patient slides down below a predetermined angle. The device operates to turn off the pump or to reverse its flow direction in order to prevent further fluid from entering the stomach and hence the esophagus. Additionally, not only does the device shut off the pump, but it includes an optional but desired modality in which it also actually withdraws residual liquid through the tube.

The sensing of patient position below a certain angle or sensing that the patient's stomach is sufficiently full is also usable to alert the attending medical staff that a patient is in an undesirable position or that feeding or medication should be stopped. Additionally, the detection of an undesirable patient angle and/or stomach fullness is also employable to automatically raise the head and/or foot portion of an adjustable bed so as to prevent or correct for downward sliding.

Accordingly, it is an object of the present invention to reduce and/or eliminate the problem of the aspiration of gastric fluid in patients connected to gastric feeding tubes.

It is also an object of the present invention to reduce and/or eliminate the problem of exposing portions of the esophagus to gastric fluids.

It is a still further object of the present invention to provide medical staff with an indication of undesired patient movement or an overfeeding condition.

It is yet another object of the present invention to provide a feedback mechanism for raising the foot or head portions of a patients bed to prevent or correct for patient sliding.

It is an additional object of the present invention to provide improved control over gastric contents as both a function of their amount, their relative amount and/or the patient angle with respect to the horizontal.

Lastly, but not limited hereto, it is an object of the present invention to provide a mechanism, which provides an indication of patient stomach content in terms of quantity.

Additional features and advantages are realized through the techniques of the present invention. Other embodiments and aspects of the invention are described in detail herein and are considered a part of the claimed invention.

The recitation herein of a list of desirable objects which are met by various embodiments of the present invention is not meant to imply or suggest that any or all of these objects are present as essential features, either individually or collectively, in the most general embodiment of the present invention or in any of its more specific embodiments.

### BRIEF DESCRIPTION OF THE DRAWINGS

The subject matter which is regarded as the invention is particularly pointed out and distinctly claimed in the concluding portion of the specification. The invention, however, both as to organization and method of practice, together with the further objects and advantages thereof, may best be understood by reference to the following description taken in connection with the accompanying drawings in which:

FIG. 1 is a side elevation view indicating the relative positions of a patient and a reclinable bed, and particularly indicating the angle of the bed;

FIG. 2 is a stylized, side elevation view of a patient showing the stomach and esophagus for a patient reclining at the angle shown in FIG. 1, as well as showing the placement of an angle sensor;

FIG. 3 is a block diagram illustrating the system and method of the present invention;

FIG. 4 is a block diagram view similar to FIG. 3 but more particularly illustrating the presence of a stomach content quantity sensor;

FIG. 5 is an enlarged view of a portion of FIG. 2, which more particularly illustrates an embodiment of the present invention employing a stomach content sensor;

FIG. 6 is a schematic diagram illustrating the use of a girth sensor for providing a fullness signal;

FIG. 7 is a schematic diagram similar to FIG. 5 but more particularly illustrating the use of a PEG tube; and

FIG. 8 is a diagram illustrating an exemplary flow control algorithm based on both patient angle and fullness sensor.

### DETAILED DESCRIPTION

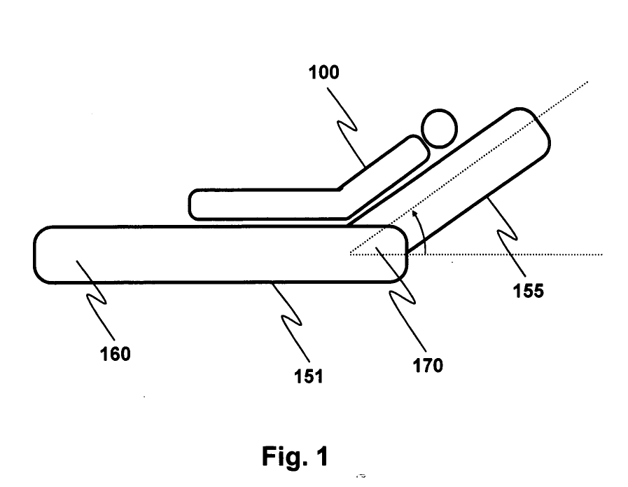

FIG. 1 illustrates the environment in which the patient angle sensor and the patient stomach content quantity devices are employed. In particular, there is shown patient 100 positioned in a reclining position on bed 151 which includes movable head portion 155 and which may also include a likewise movable foot portion 160 which is employed either for patient comfort or for elevation of the lower extremities. It is also seen that the patient is reclining at angle (170) with respect to the horizontal. Reference to the horizontal is employed herein for measurement and determination of improper angle since the “horizontal” is really determined by gravity and it is gravity that is the principal driver of gastric fluid into the esophagus and beyond.

It is noted herein that the angle shown in FIG. 1 is the angle of the adjustable head portion of the bed with respect to the horizontal portion of the bed. Even though the illustration suggests it, FIG. 1 does not reflect the position of a patient who has slid down in the bed. It should also be noted that the beds of concern herein may also be equipped with adjustable foot portion 160 as well as with adjustable head portion 155 and mid-portion 150. In fact, if it is detected that patient 100 is sliding down in the bed, the adjustable foot portion of the bed may be raised to prevent further sliding and/or to correct for prior sliding. Other portions of the bed may be adjusted as well in various relative motions designed to control the patient's angle with respect to the horizontal. This is an optional feature of the present invention.

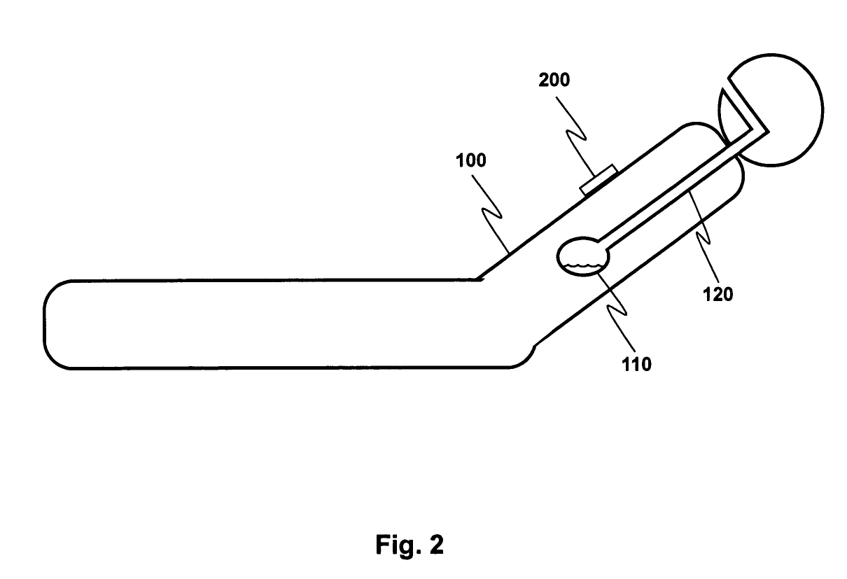

FIG. 2 provides a greater detail of the situation being considered with respect to patient 100 and the problem of patient angle. Basic human anatomy teaches that stomach 110 is connected to esophagus 120. It is easily seen that if the patient's angle is low, that is, if the patient's torso is closer to a horizontal position, stomach contents can enter esophagus 120 simply by gravity flow. The problems associated with this flow are discussed above, but, needless to say, it is not a desirable situation.

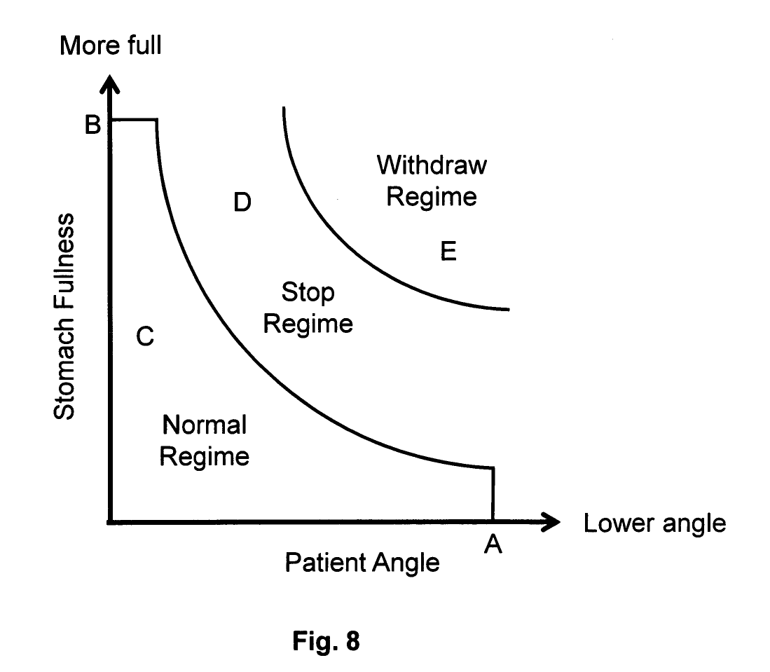

Additionally, FIG. 2 illustrates the placement of sensor 200. Sensor 200 is preferably placed on the chest of patient 200. It is affixed to the patient or to the patient's clothing by any convenient means. For short term use adhesive material on one side of sensor 200 holds it in place. For use with clothing or gowns, a wider range of options is available for affixing the sensor, including pins, elastic bands and Velcro™. Sensor 200 comprises any convenient mechanism for sensing angle. At its simplest it comprises a mercury filled insulative container with electrical contacts being closed when it makes contact with the mercury. The interior shape of the container is such that the mercury comes in contact with the contacts at a predetermined angle. The sensor may also include adjustable exterior flaps to provide a selectable angle. It is noted, however, that there is a wide range of sensors and sensor technology which may be employed. For example, one could employ a ball or other sliding or rolling interior object which either makes electrical contact or which is of sufficient weight to cause switch contacts to close. Additionally, the interior moving object may be employed to interrupt light falling on a photocell. Magnetic or other optical sensors may be employed as well. In fact, any device which implements the generation of an electrical or even electromagnetic signal based on dependence on an angle is employable. As indicated, sensor 200 may even comprise a wireless device which transmits an activation signal to pump control 220. More sophisticated sensors 200 which actually provide a signal indicative of the actual angle, as opposed to the angle merely exceeding a threshold value are also employed in the present invention. Such devices are relevant to the control modality suggested by FIG. 8. With a more sophisticated indication of angle being provided, it is then possible to provide an early warning indication of a patient sliding downward. In such cases, the alarm to patient or staff is variable in intensity depending on the angular degree sensed.

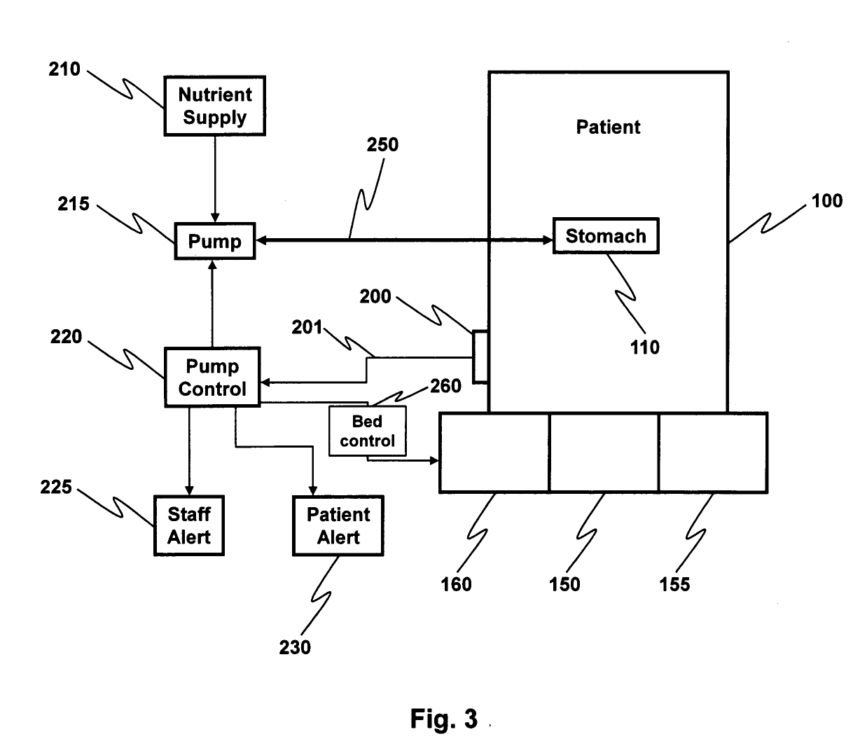

A solution to the aspiration problem based solely on patient angle is shown in greater detail in FIG. 3. In particular, angle sensor 200, which is affixed to patient 100, sends a signal via wire 201 to pump control 220 which, in normal operation, sends nutrient materials from supply 210 to stomach 110 of patient 100. If patient 100 slides down in bed 150 to an undesired, predetermined angle or range of angles, angle sensor 200 signals pump control 220 to shut off the supply of nutrient or other material to stomach 110. Additionally, the system is provided with an optional feature in which gastric fluid is actually pulled back into gastric tube 250. In this regard, note the two directions indicated for tube 250. It is noted that if angle sensor 200 produces a signal that is transmitted wirelessly, wire 201 is not needed.

It is also seen that the signal from angle sensor 200 is also capable of providing an audible or visual signal 225 to hospital staff members to alert them that patient 100 has slid down into bed 150 to an undesirable and possibly unsafe position. Pump control 220 may also be used to supply an audible, visual or vibratory signal 230 to patient 100 as a mechanism for immediate correction by the patient himself or herself, if possible. This same signal from sensor 200 may also be used to control bed 151. In particular, in conjunction with bed control unit 260 (shown only in FIG. 3 for convenience), angle sensor 200 is also seen to be capable of providing an actuation signal to cause foot portion 160 of bed 151 to rise so as to forestall further sliding and/or to correct for previous sliding.

In the discussions above, it is assumed that nutrients are provided through a gastric tube via a pump which acts as a positive control element in the system. However, it is noted that it is also possible that nutrient supply 210 may be positioned above the patient so that it is supplied by gravitational action. In this case, the role of “pump” 220 is less “active” in that it operates not so much as a pump but as a valve to control the rate of flow. In such an arrangement the optional feature of pump reversal is not available. However, apart from this drawback, the angle sensing aspects and the fullness sensing aspects are equally capable of operating with gravity flow systems.

Pump control 220 is provided by any convenient mechanism. Application specific integrated circuit (ASIC) chips may be employed in pump control 220 or off-the shelf control components may be used, or pump control 220 may be implemented via any standard microprocessor or microcontroller. An exemplary control algorithm based on sensed patient angle and patient stomach content level is shown in FIG. 8.

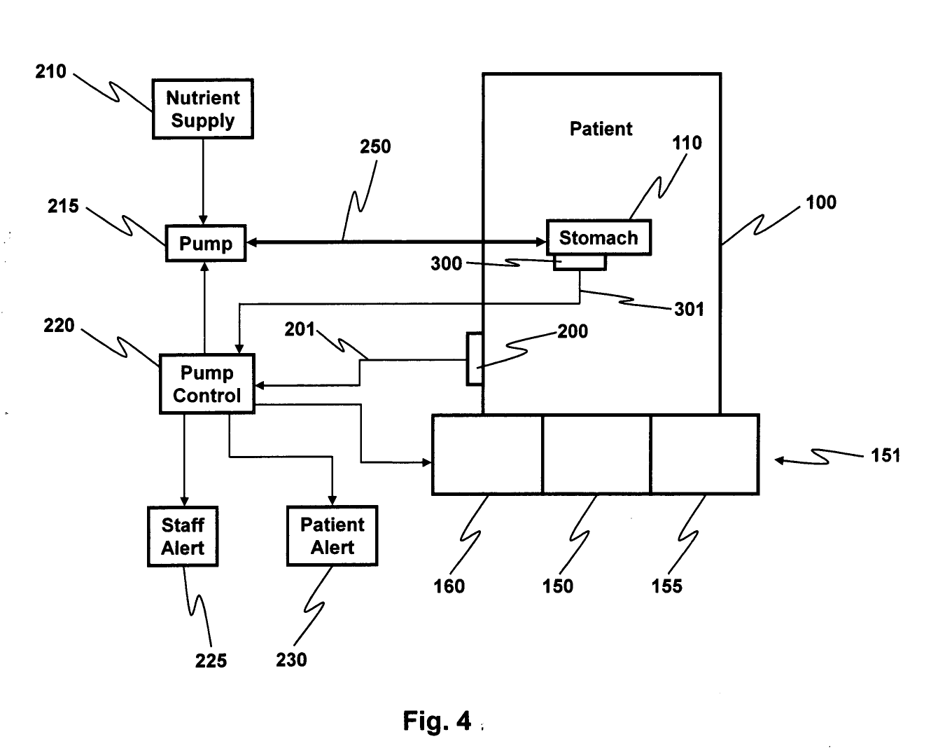

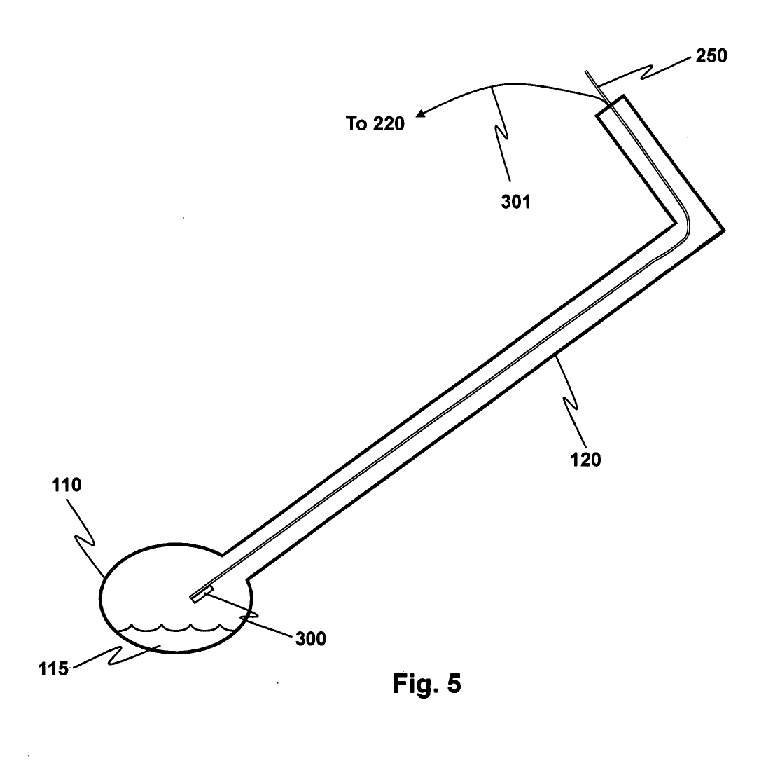

FIG. 4 is similar to FIG. 3 but it more particularly illustrates the presence of an additional mechanism which is capable of providing an indication of the quantity of material within the stomach at any given time. In particulate, one form of fullness sensor 300 is disposed at the end of feeding tube 250 as shown in FIG. 5. When implemented in this fashion, fullness sensor 300 has connected thereto signal wire or cable 301 which is typically disposed alongside feeding tube 250 or may be manufactured along with it as an integral assembly. Wire or cable 301 is provided to pump control 220 to be used, either alone or in conjunction with a signal from angle sensor 200, to control the flow of fluid in feeding tube 250, either stopping it, or in some cases, actually reversing the flow.

Fullness sensor 300, as shown in FIG. 5 may comprise an electrical circuit whose properties change when in contact with gastric fluid 115. Fullness sensor 300 may also respond to being in contact with any liquid; it may respond to being in contact with a liquid of a certain acidity; or fullness sensor 300 may respond to the level of liquid present. Additionally, fullness sensor 300 may also include ultrasonic transmission and receiving components which produce a signal which is proportional to or a function of unoccupied gastric volume. In this way, if a known volume of fluid is introduced into the stomach in a known amount of time, ultrasonic fullness sensor 300 provides “before” and “after” signals which can be used to indicate the change in stomach volume as a percentage which occurs as the result of the input of a known volume in a known amount of time. In this way, stomach volume can be calculated and the sensor can be calibrated accordingly. Fullness sensor 300 may also comprise a pressure transducer which responds to elevated levels of gas pressure within the stomach.

Exterior ultrasound measurements produced using readily available equipment may also be employed as a mechanism for determining fullness and the need to either stop or withdraw fluid. This approach, however, typically has the disadvantage of requiring human intervention and is harder to automate.

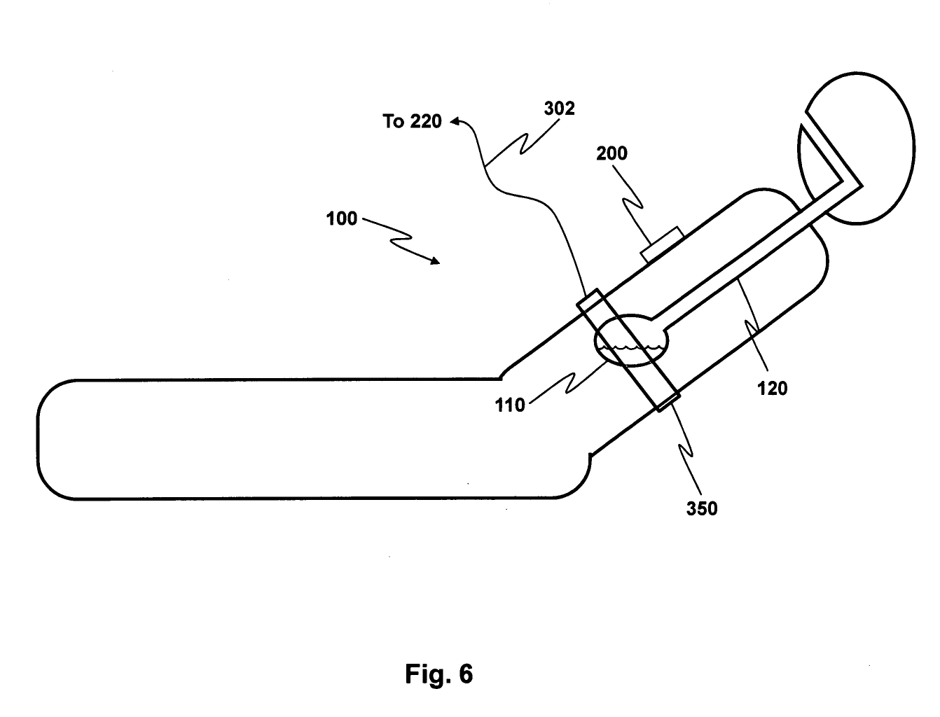

FIG. 6 illustrates the situation in which girth sensor 350 is employed as a mechanism for determining stomach fullness and/or changes in stomach fullness. Girth sensor 350 is disposed about the patient's abdomen as shown and lead 302 is supplied to pump control 220. In the event that girth sensor 350 includes a wireless transmission device, electrical conductor 302 is not necessary.

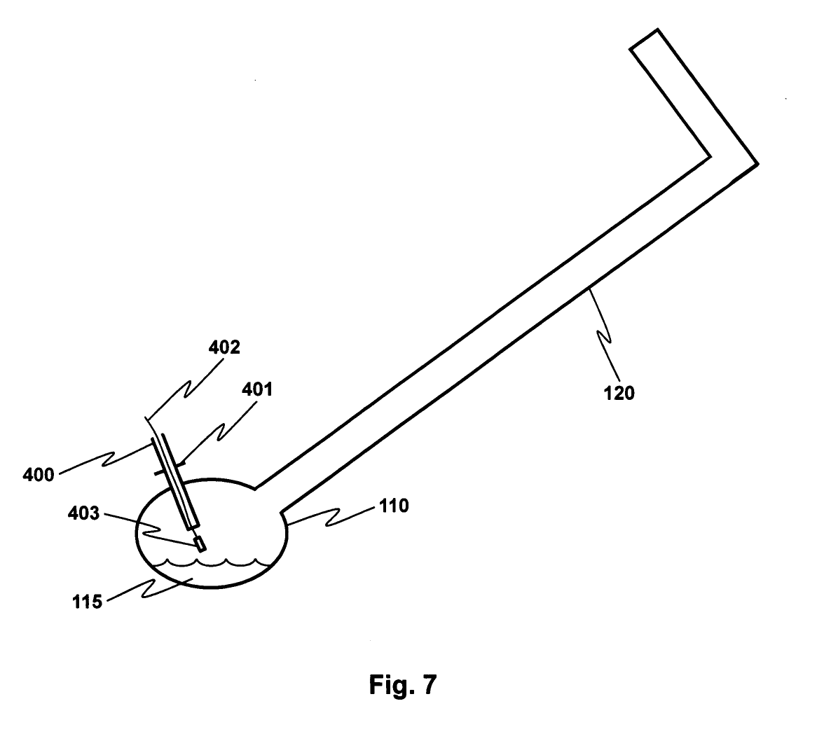

FIG. 7 illustrates the use of the present invention when, instead of a nasogastric tube, PEG tube 400 is employed. Such tubes typically include collar portion 401 which is disposed against the abdomen and is affixed thereto in a sealed fashion to guard against providing a passage for infection. Fullness sensor 403 is disposed through PEG tube 400 and is coupled externally through electrical conductor 402.

FIG. 8 represents an exemplary algorithm for pump control and/or stoppage control (the latter being especially in the case of a gravity driven nutrient supply) based jointly on patient angle and patient stomach fullness. In the case of each variable, it is seen that there is a point reached where some action is taken such as when the patient angle gets too low (point A in FIG. 8) or when the patient's stomach contents become too full (point B), this latter point being particularly desirable in the implementation of a method designed to keep stomach contents out of the esophagus, independent of patient angle. Also shown in FIG. 8 is region C which illustrates normal operation in a region of relatively high patient angle and low stomach contents. As these variables change in a direction away from the illustrated origin, control enters a control regime D in which feeding or nutrition flow is stopped. Further excursions of these variables in a direction away from the indicated origin result in flow control entering region E characterized not just by flow stoppage but by flow reversal. As should be fully appreciated, variations of the regions illustrated in FIG. 8 are not only possible to achieve specific purposes in particular patients but it is also easily possible to implement any diagram such as that shown using microprocessors with the given curves stored in its memory in a number of convenient forms.

While the invention has been described in detail herein in accordance with certain preferred embodiments thereof, many modifications and changes therein may be effected by those skilled in the art. Accordingly, it is intended by the appended claims to cover all such modifications and changes as fall within the true spirit and scope of the invention.

What is claimed is:

1. A device to prevent aspiration of gastric fluids in a patient being fed or medicated through a gastric tube, said device comprising:
an angle sensor affixable to said patient, said sensor being capable of providing an electrical signal indicative of the patient's torso being angularly positioned below a threshold angle with respect to the horizontal;
a stomach fullness sensor for monitoring said patient's stomach content, said sensor being capable of providing an electrical signal indicative of patient stomach content quantity; and
an electrical control circuit for receiving at least one of said signals and for controlling flow in said gastric tube as a function of at least one of said signals.

2. The device of claim 1 in which said electrical control circuit is capable of controlling a pump so as to reverse flow in said gastric tube.

3. The device of claim 1 in which said fullness sensor provides an electrical signal indicative of stomach content as measured by patient girth.

4. The device of claim 1 in which said fullness sensor provides an electrical signal indicative of stomach content as measured by internal stomach pressure.

5. The device of claim 1 in which said fullness sensor provides an electrical signal indicative of stomach content as measured by stomach content level.

5. The device of claim 1 in which said fullness sensor provides an electrical signal indicative of stomach content as measured by stomach acidity.

6. The device of claim 1 in which said electrical control circuit receives both of said signals.

7. The device of claim 1 in which said control circuit actuates an alarm to alert staff.

8. The device of claim 1 in which said control circuit actuates an alarm to alert said patient.

9. The device of claim 1 in which said threshold angle is adjustable.

10. The device of claim 1 in which said electrical control circuit operates to raise a head portion of said patient's bed.

11. The device of claim 1 in which said electrical control circuit operates to raise a foot portion of said patient's bed.

12. The device of claim 1 in which said angle sensor includes a wireless transmitter to supply said signal to said control circuit which includes a receiver for said signal.

13. The device of claim 1 in which said fullness sensor includes a wireless transmitter to supply said signal to said control circuit which includes a receiver for said signal.

14. A device to prevent aspiration of gastric fluids in a patient receiving fluid through a gastric tube, said device comprising:
a stomach fullness sensor for monitoring said patient's stomach content, said sensor being capable of providing an electrical signal indicative of patient stomach content quantity; and
an electrical control circuit for receiving said patient stomach content signal and for stopping flow in said tube, as a function of said content level.

15. A gastric feeding system for a patient, said system comprising:
a nutrient supply reservoir;
a pump connected to said supply reservoir:
a gastric tube for delivering nutrient from said nutrient supply reservoir to said patient;
an electrical circuit for controlling said pump; and
a stomach fullness sensor for monitoring said patient's stomach content, said sensor being capable of providing an electrical signal indicative of patient stomach content quantity to said electrical circuit, whereby fluid flow in said gastric tube may be stopped or reversed upon an indication of stomach content above a threshold value.

16. A method for ameliorating the problem of stomach content aspiration for a patient being fed or medicated through a gastric tube, said method comprising the step of:
stopping fluid flow through said gastric tube upon detecting that said patient's stomach content is above a threshold value.

17. A method for ameliorating the problem of stomach content aspiration for a patient being fed or medicated through a gastric tube, said method comprising the step of:
reversing fluid flow through said gastric tube upon detecting that said patient's stomach content is above a threshold value.
